(19) United States
(12) Patent Application Publication
(10) Pub. No.: US 2024/0378937 A1
(43) Pub. Date: Nov. 14, 2024

(54) BANKNOTE STERILIZATION SYSTEM

(71) Applicant: NIPPON TELEGRAPH AND TELEPHONE CORPORATION, Tokyo (JP)

(72) Inventors: Asuka MIYAKE, Musashino-shi, Tokyo (JP); Hitoshi SESHIMO, Musashino-shi, Tokyo (JP); Ryuji YAMAMOTO, Musashino-shi, Tokyo (JP); Tomohiro TANIGUCHI, Musashino-shi, Tokyo (JP); Ayako IWAKI, Musashino-shi, Tokyo (JP); Satoshi NARIKAWA, Musashino-shi, Tokyo (JP); Takahito KIRIHARA, Musashino-shi, Tokyo (JP); Kazuhide NAKAJIMA, Musashino-shi, Tokyo (JP); Takashi MATSUI, Musashino-shi, Tokyo (JP); Yuto SAGAE, Musashino-shi, Tokyo (JP); Chisato FUKAI, Musashino-shi, Tokyo (JP); Nobutomo HANZAWA, Musashino-shi, Tokyo (JP)

(73) Assignee: NIPPON TELEGRAPH AND TELEPHONE CORPORATION, Tokyo (JP)

(21) Appl. No.: 18/689,034

(22) PCT Filed: Sep. 24, 2021
(86) PCT No.: PCT/JP2021/035063

§ 371 Date: Mar. 4, 2024

(51) Int. Cl. G07D 11/16; A61L 2/10; G07D 11/12

(52) U.S. Cl. CPC G07D 11/16; A61L 2/10; G07D 11/12

(57) ABSTRACT

An object of the present invention is to provide a banknote disinfection system configured to disinfect banknotes in a general-purpose banknote handling device without making a significant change in the structure of the general-purpose device.

The banknote disinfection system 300 includes a conveyance mechanism configured to convey a banknote 50 using a plurality of rollers 12 and an optical fiber 20 configured to emit ultraviolet light to a surface of the banknote 50 conveyed by the conveyance mechanism. An optical fiber is thin, has many length options, and is flexible. With these features of the optical fiber, the optical fiber can be routed inside of, for example, an automatic teller machine to irradiate the banknotes moving inside the device with ultraviolet light.

### TECHNICAL FIELD

The present disclosure relates to a banknote disinfection system that performs sterilization and inactivation of viruses on banknotes using ultraviolet light.

### BACKGROUND ART

To prevent infectious diseases or the like, there has been a growing demand for a system that performs sterilization and inactivation of viruses using ultraviolet light. In the present embodiment, the word “disinfection” includes sterilization and inactivation of viruses.

A banknote disinfector for disinfecting banknotes is known (see, for example, Non Patent Literature 1). This disinfector performs disinfection by emitting intense ultraviolet light (UV-C) to both sides of the banknotes one by one.

### CITATION LIST

### Non Patent Literature

Non Patent Literature 1: Hitachi-Omron Terminal Solutions, Corp. website (https://www.hitachi-omron-ts.co.jp/news/2020/202011-001.html), searched on Apr. 19, 2021

### SUMMARY OF INVENTION

### Technical Problem

Since the size of the current ultraviolet lamp is large, devices for disinfecting banknotes have to be such a specialized large one as described above. For this reason, the banknote disinfector has been developed only for banks and the like in which a large number of banknotes are disinfected, and there is no banknote disinfector available for ordinary people. In this regard, it is difficult to disinfect banknotes circulating in daily lives.

The present invention has been made in view of the foregoing, and it is an object of the present invention to provide a banknote disinfection system configured to disinfect banknotes in a general-purpose banknote handling device without making a significant change in the structure of the general-purpose device.

### Solution to Problem

In order to achieve the object above, a banknote disinfection system according to the present invention includes an optical fiber as a small irradiation unit configured to emit ultraviolet light.

Specifically, the banknote disinfection system according to the present invention includes a conveyance mechanism configured to convey a banknote using a plurality of rollers and an optical fiber configured to emit ultraviolet light to a surface of the banknote conveyed by the conveyance mechanism.

An optical fiber is thin, has many length options, and is flexible. With these features, the optical fiber can be routed inside of, for example, an automatic teller machine (ATM) to irradiate the banknotes moving inside the device with ultraviolet light. Since an ultraviolet lamp or an LED lamp has a diameter of several millimeters to several centimeters, performing irradiation using these lamps in the ATM requires a significant change in the design of the current ATM. The optical fiber, however, can be routed through gaps in the current ATM, and this system can be introduced without making a significant change in the current device design.

The present invention can, therefore, provide a banknote disinfection system configured to disinfect banknotes in a general-purpose banknote handling device without making a significant change in the structure of the general-purpose device.

The optical fiber of the banknote disinfection system according to the present invention has an end directed to the surface of the banknote conveyed by the conveyance mechanism, and is configured to emit propagating ultraviolet light from the end.

The optical fiber of the banknote disinfection system according to the present invention is disposed with a central axis being parallel to the surface of the banknote, and is configured to emit part of propagating ultraviolet light from a side surface.

The banknote disinfection system according to the present invention further includes a storage configured to store the banknote, and the conveyance mechanism takes out the banknote in the storage at certain time to bring the banknote into irradiation with ultraviolet light from the optical fiber.

This configuration enables the banknotes stored in the device to be periodically circulated to an ultraviolet irradiation area for disinfection.

The banknote disinfection system according to the present invention further includes a storage configured to store the banknote, and a second optical fiber configured to emit ultraviolet light to the banknote in the storage. This configuration enables disinfection of banknotes stored in the storage.

In this case, the second optical fiber has an end directed to the surface of the banknote stored in the storage, and is configured to emit propagating ultraviolet light from the end. The second optical fiber may be disposed at the bottom of the storage and configured to emit part of the propagating ultraviolet light from a side surface.

Note that each of the above inventions can be combined in any possible manner.

### Advantageous Effects of Invention

The present invention can provide a banknote disinfection system configured to disinfect banknotes in a general-purpose banknote handling device without making a significant change in the structure of the general-purpose device.

### BRIEF DESCRIPTION OF DRAWINGS

FIG. 1 is a diagram illustrating an example in which a banknote disinfection system according to the present invention is installed in an ATM.

FIG. 2 is a diagram illustrating the banknote disinfection system according to the present invention.

FIG. 3 is a diagram illustrating the banknote disinfection system according to the present invention.

FIG. 4 is a diagram illustrating the banknote disinfection system according to the present invention.

FIG. 5 is a diagram illustrating the banknote disinfection system according to the present invention.

FIG. 6 is a diagram illustrating the banknote disinfection system according to the present invention.

### DESCRIPTION OF EMBODIMENTS

The following describes embodiments of the present invention with reference to the accompanying drawings. The embodiments described below are examples of the present invention, and the present invention is not limited to the following embodiments. The same reference signs in the present description and the drawings denote the same components.

### First Embodiment

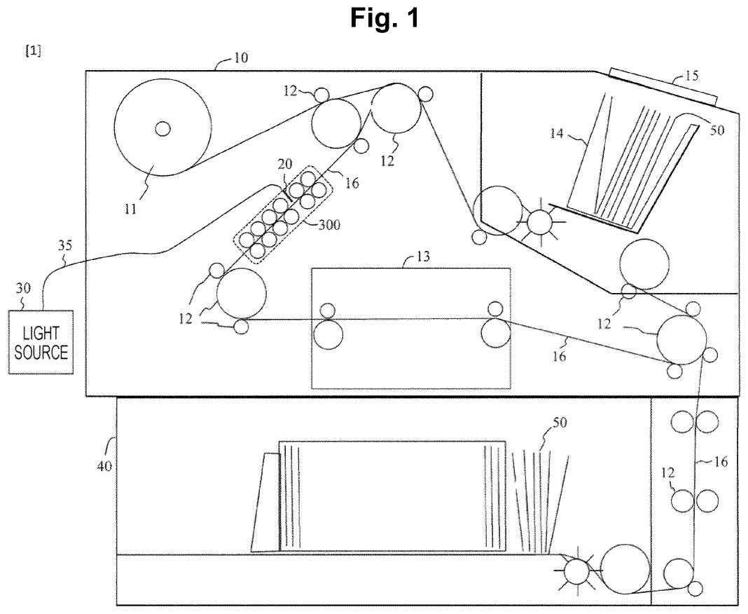

FIG. 1 is a diagram illustrating an example in which a banknote disinfection system 300 according to the present embodiment is installed in an ATM 10. The ATM 10 includes a deposit and withdrawal port 14 for depositing and withdrawing banknotes 50. At an upper surface of the deposit and withdrawal port 14, a shutter 15 is provided that opens and closes automatically. A functional unit 13 is a certain functional unit configured to, for example, verify the authenticity of the banknotes and specify the types of the banknotes. A temporary storage 11 is a mechanism that temporarily stores the banknotes 50 at the time of deposition. The banknotes 50 are conveyed by rollers 12 disposed along a conveyance path 16 inside the ATM 10.

The banknotes 50 inserted into the deposit and withdrawal port 14 at the time of deposition are conveyed to the temporary storage 11 via the conveyance path 16 and the functional unit 13. The functional unit 13 identifies the banknotes and counts the amount of the deposited banknotes 50. When the user confirms the counted amount and gives an instruction to deposit, the banknotes 50 are taken out from the temporary storage 11 and conveyed to a storage (drawer) 40 via the conveyance path 16 and the functional unit 13. At the time of withdrawal, the banknotes 50 are taken out from the storage 40 and conveyed to the deposit and withdrawal port 14 via the conveyance path 16 and the functional unit 13.

The banknote disinfection system 300 according to the present embodiment is installed in part of the conveyance path 16. The banknote disinfection system 300 includes a conveyance mechanism configured to convey a banknote 50 using a plurality of rollers 12 and an optical fiber 20 configured to emit ultraviolet light to a surface of the banknote 50 conveyed by the conveyance mechanism. A light source 30 configured to output ultraviolet light is installed outside the ATM 10, and the ultraviolet light output from the light source 30 is supplied to the optical fiber 20 through an optical transmission line 35.

The banknote 50 conveyed on the conveyance path 16 is disinfected while passing through the banknote disinfection system 300. The conveyance mechanism may take out the banknote 50 in the storage 40 at certain time to bring the banknote 50 into irradiation with ultraviolet light emitted from the optical fiber 20. This configuration enables the banknotes 50 stored in the storage 40 to be periodically circulated to an ultraviolet irradiation area for disinfection.

The conveyance mechanism may be separate from the conveyance mechanism (rollers 12) of the ATM 10, or may be configured to use the rollers 12 of the ATM 10. In other words, the optical fiber 20 may be disposed in a gap between the rollers 12 of the ATM 10. The installation location of the banknote disinfection system 300 is not limited to the location illustrated in FIG. 1. Since the optical fiber 20 has a small diameter and is flexible, the optical fiber 20 can be routed through a gap. Thus, the banknote disinfection system 300 can be installed in a narrow gap in the ATM 10 that has already been installed.

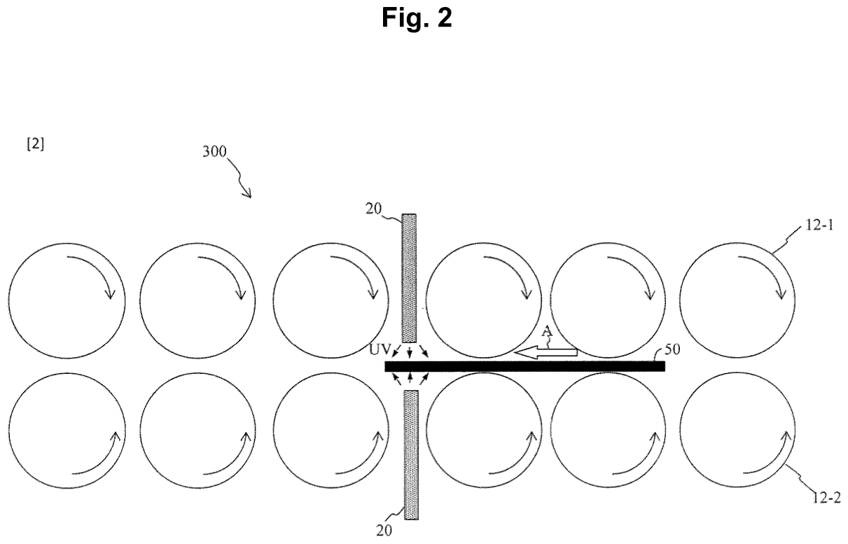

The following describes a specific disposition example of the optical fiber 20 with reference to FIGS. 2 to 5.

### Example 1

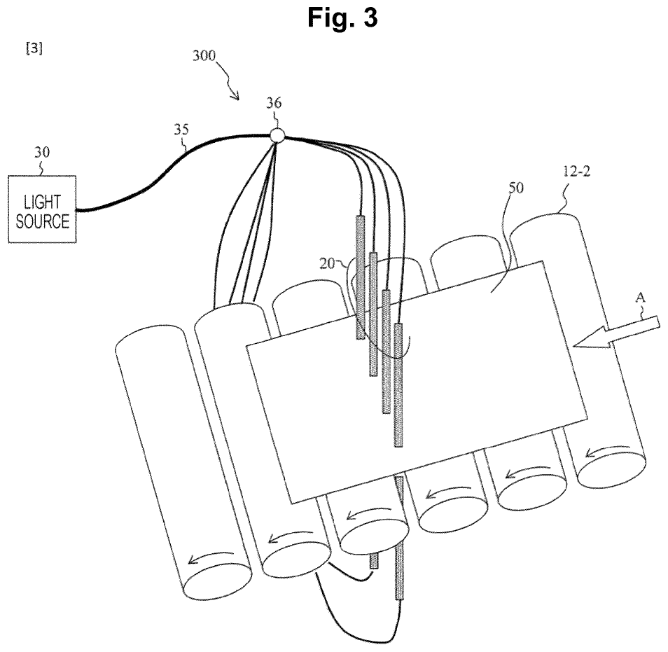

FIGS. 2 and 3 are diagrams illustrating an example in which the end of the optical fiber 20 is directed to a surface of a banknote 50 conveyed by the conveyance mechanism (rollers 12), and propagating ultraviolet light is emitted from the end of the optical fiber 20. The banknote 50 is sandwiched between upper rollers 12-1 and lower rollers 12-2, and the banknote 50 is conveyed as the rollers rotate. Arrow A indicates the direction in which the banknote 50 is conveyed.

FIG. 2 is a diagram of the banknote disinfection system 300 seen in the axial direction of the rollers 12. FIG. 3 is a diagram of the banknote disinfection system 300 seen at an oblique angle from above, and the upper rollers 12-1 are not illustrated for ease of explanation. The optical fibers 20 are disposed with their ends located in a gap between adjacent rollers 12-1 and a gap between adjacent rollers 12-2, and emit ultraviolet light from the ends. The ultraviolet light is transmitted from the light source 30 through the optical transmission line 35, branched at an optical branching unit 36, and supplied to the optical fibers 20.

Since ultraviolet light is emitted from the ends of the optical fibers 20, the banknote 50 conveyed by the rollers 12 is disinfected by the ultraviolet light as the banknote 50 passes through between the optical fibers 20. The banknote disinfection system 300 may detect in advance the passing of the banknote 50 through the rollers 12 and send a command to the light source 30 to supply ultraviolet light.

In this example, four optical fibers 20 are disposed above the banknote 50 and the other four optical fibers 20 are disposed below the banknote 50, but the number of optical fibers 20 to be disposed is optional.

### Example 2

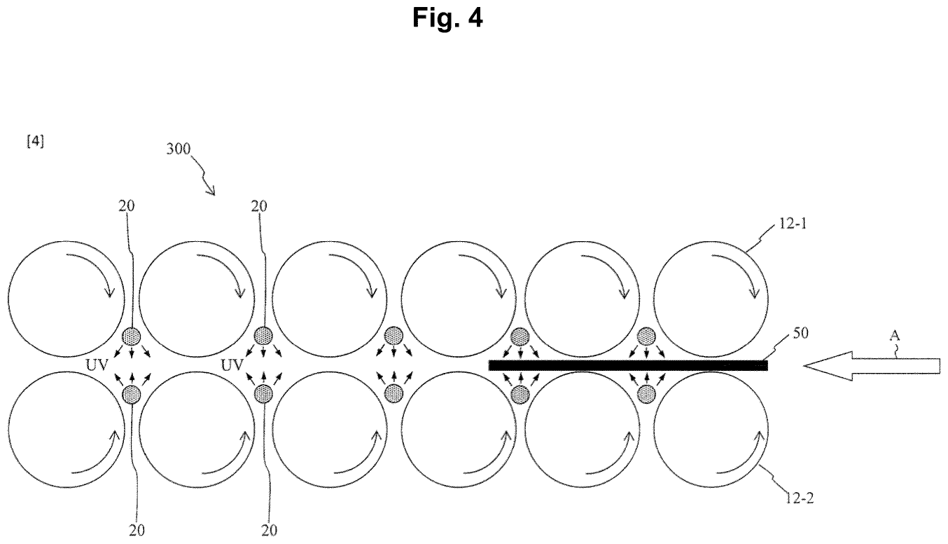

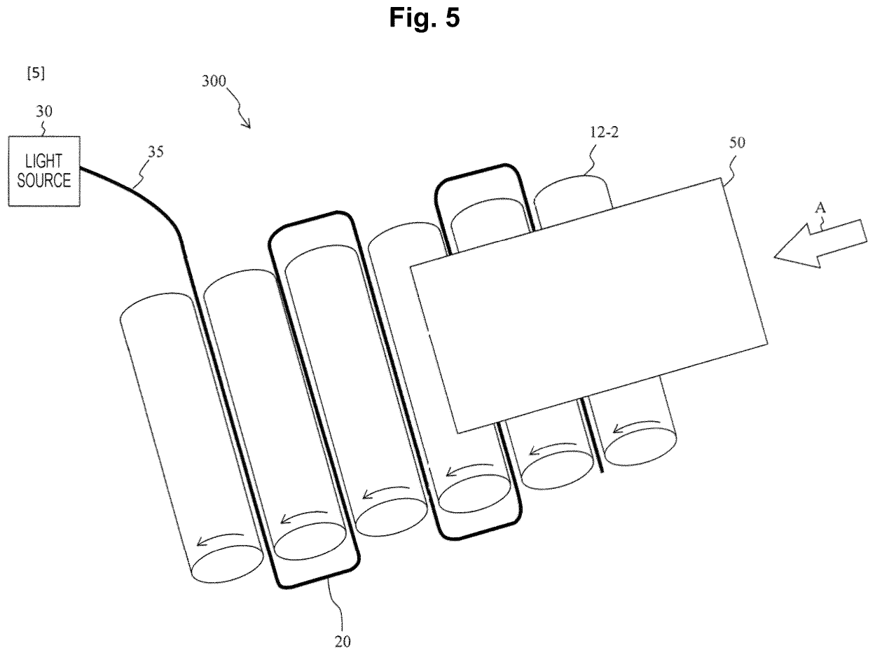

FIGS. 4 and 5 are diagrams illustrating an example in which the optical fiber 20 is disposed with its central axis being parallel to the surface of the banknote 50 and emits part of the propagating ultraviolet light from a side surface. The banknote 50 is sandwiched between upper rollers 12-1 and lower rollers 12-2, and the banknote 50 is conveyed as the rollers rotate. Arrow A indicates the direction in which the banknote 50 is conveyed.

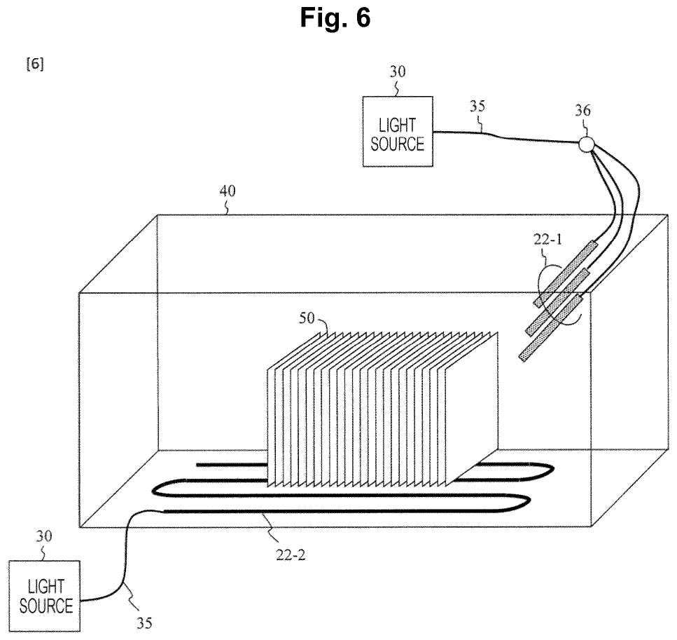

FIG. 4 is a diagram of the banknote disinfection system 300 seen in the axial direction of the rollers 12. FIG. 5 is a diagram of the banknote disinfection system 300 seen at an oblique angle from above, and the upper rollers 12-1 are not illustrated for ease of explanation. The optical fiber 20 is disposed in the gaps between adjacent rollers 12-1 and in the gaps between adjacent rollers 12-2 with the axis being parallel to the axis of the rollers 12. The optical fiber 20 is a side-emitting optical fiber. As illustrated in FIGS. 5 and 6, it is preferred that a single optical fiber 20 is routed through between the rollers 12. The ultraviolet light is transmitted from the light source 30 through the optical transmission line 35 and supplied to the optical fiber 20.

Since ultraviolet light is emitted from the side surface of the optical fiber 20, the banknote 50 conveyed by the rollers 12 is disinfected by the ultraviolet light as the banknote 50 passes through between the rollers 12-1 and the rollers 12-2. The banknote disinfection system 300 may detect in advance the passing of the banknote 50 through the rollers 12 and send a command to the light source 30 to supply ultraviolet light.

In this example, one optical fiber 20 is disposed above the banknote 50 and one optical fiber 20 is disposed below the banknote 50 and each is routed between the rollers, but a plurality of optical fibers 20 may be provided and disposed between the rollers.

### Second Embodiment

The banknote disinfection system 300 disposed in a main body of the ATM 10 has been described with reference to FIGS. 1 to 5. In the present embodiment, another example is described with reference to FIG. 6 in which a structure for emitting ultraviolet light is also disposed in the storage 40 attached to the ATM 10. In other words, a banknote disinfection system according to the present embodiment further includes the storage 40 that stores banknotes 50, and a second optical fiber 22 configured to emit ultraviolet light to the banknotes 50 in the storage 40.

FIG. 6 illustrates second optical fibers 22-1 each having an end directed to a surface of banknotes 50 stored in the storage 40 and configured to emit propagating ultraviolet light from the end. The ends of the optical fibers 22-1 are inserted into the storage 40 and directed toward the banknotes 50 stored therein. The ultraviolet light is transmitted from a light source 30 through an optical transmission line 35, branched at an optical branching unit 36, and supplied to the optical fibers 22-1. The optical fibers 22-1 emit ultraviolet light to the banknotes 50 from their ends.

Since ultraviolet light is emitted from the ends of the optical fibers 22-1, the banknotes 50 are disinfected by the ultraviolet light while being stored in the storage 40. The banknote disinfection system 300 may irradiate the banknotes 50 with ultraviolet light when the banknotes 50 are stored in or taken out of the storage 40.

In this example, three optical fibers 22-1 are disposed for the banknotes 50 in the storage 40, but the number of optical fibers 22-1 to be disposed is optional. The optical fibers 22-1 may be disposed with their ends being located at a banknote inlet and outlet port of the storage 40.

FIG. 6 also illustrates a second optical fiber 22-2 disposed at the bottom of the storage 40 and configured to emit part of the propagating ultraviolet light from the side surface. The optical fiber 22-2 is a side-emitting optical fiber. As illustrated in FIG. 6, it is preferred that a single optical fiber 22-2 is routed on the bottom of the storage 40. The ultraviolet light is transmitted from the light source 30 through the optical transmission line 35 and supplied to the optical fiber 22-2.

Since ultraviolet light is emitted from the bottom of the storage 40 to the banknotes 50, the banknotes 50 are disinfected by the ultraviolet light while being stored in the storage 40. In addition, when there is no banknote 50 in the storage 40, the irradiation of the ultraviolet light may be stopped.

### Other Embodiments

In the embodiments above, an example is described in which the banknote disinfection system 300 is installed in the ATM 10. The banknote disinfection system 300 can be installed in a general-purpose device other than the ATM 10, thereby enabling disinfection of banknotes circulating in daily lives by ultraviolet light irradiation. Installing the banknote disinfection system 300 in a cash register of a store allows for more frequent disinfection of banknotes.

### REFERENCE SIGNS LIST

10 ATM
11 Temporary storage
12, 12-1, 12-2 Roller
13 Functional unit
14 Deposit and withdrawal port
15 Shutter
16 Conveyance path
20 Optical fiber
22, 22-1, 22-2 Second optical fiber
30 Light source
35 Optical transmission line
36 Optical branching unit
40 Storage
50 Banknote
300 BANKNOTE DISINFECTION SYSTEM

1. A banknote disinfection system comprising:
a conveyance mechanism configured to convey a banknote using a plurality of rollers; and
an optical fiber configured to emit ultraviolet light to a surface of the banknote conveyed by the conveyance mechanism.

2. The banknote disinfection system according to claim 1, wherein the optical fiber has an end directed to the surface of the banknote conveyed by the conveyance mechanism, and is configured to emit propagating ultraviolet light from the end.

3. The banknote disinfection system according to claim 1, wherein the optical fiber is disposed with a central axis being parallel to the surface of the banknote, and is configured to emit part of propagating ultraviolet light from a side surface.

4. The banknote disinfection system according to claim 1, further comprising a storage configured to store the banknote, wherein
the conveyance mechanism takes out the banknote in the storage at certain time to bring the banknote into irradiation with ultraviolet light from the optical fiber.

5. The banknote disinfection system according to claim 1, further comprising:
a storage configured to store the banknote; and
a second optical fiber configured to emit ultraviolet light to the banknote in the storage.

6. The banknote disinfection system according to claim 5, wherein the second optical fiber has an end directed to the surface of the banknote stored in the storage, and is configured to emit propagating ultraviolet light from the end.

7. The banknote disinfection system according to claim 5, wherein the second optical fiber is disposed at a bottom of the storage, and is configured to emit part of propagating ultraviolet light from a side surface.
